(19) United States
(12) Patent Application Publication
(10) Pub. No.: US 2025/0082292 A1
(43) Pub. Date: Mar. 13, 2025

(54) SYSTEM AND METHOD FOR TARGETED OBJECT ENHANCEMENT TO GENERATE SYNTHETIC BREAST TISSUE IMAGES

(71) Applicant: Hologic, Inc., Marlborough, MA (US)

(72) Inventors: Haili CHUI, Fremont, CA (US); Liyang WEI, San Jose, CA (US); Jun GE, Cupertino, CA (US); Nikolaos GKANATSIOS, Newtown, CT (US)

(73) Assignee: Hologic, Inc., Marlborough, MA (US)

(21) Appl. No.: 18/776,823

(22) Filed: Jul. 18, 2024

Related U.S. Application Data
(63) Continuation of application No. 17/847,720, filed on Jun. 23, 2022, now Pat. No. 12,070,349.
Application No. 17/847,720 is a continuation of application No. 16/497,767, filed on Sep. 25, 2019, now Pat. No. 11,445,993.
(60) Provisional application No. 62/479,036, filed on Mar. 30, 2017.

(51) Int. Cl. A61B 6/00; A61B 6/02; A61B 6/46; A61B 6/50; G06T 5/00; G06T 5/50; G06T 7/00

(52) U.S. Cl. CPC A61B 6/5217; A61B 6/463; A61B 6/469; A61B 6/502; A61B 6/5205; A61B 6/5235; G06T 5/00; G06T 5/50; G06T 7/0012; A61B 6/025; G06T 2200/24; G06T 2207/10112; G06T 2207/20221; G06T 2207/30068

(57) ABSTRACT

A method for processing breast tissue image data includes obtaining image data of a patient's breast tissue, processing the image data to generate a set of image slices, the image slices collectively depicting the patient's breast tissue; feeding image slices of the set through each of a plurality of object-recognizing modules, each of the object-recognizing modules being configured to recognize a respective type of object that may be present in the image slices; combining objects recognized by the respective object-recognizing modules to generate a synthesized image of the patient's breast tissue; and displaying the synthesized image.

### CROSS-REFERENCE TO RELATED APPLICATIONS

This application is a continuation of U.S. patent application Ser. No. 17/847,720, filed Jun. 23, 2022, which is a continuation of U.S. patent application Ser. No. 16/497,767, now U.S. Pat. No. 11,445,993, filed Sep. 25, 2019, which is a National Stage Application of PCT/US2018/024913, filed Mar. 28, 2018, which claims the benefit of U.S. Provisional Application No. 62/479,036, filed Mar. 30, 2017, the entire disclosures of which are incorporated herein by reference in their entireties. To the extent appropriate, a claim of priority is made to each of the above disclosed applications.

### FIELD

The presently disclosed inventions relate generally to breast imaging techniques such as tomosynthesis, and more specifically to systems and methods for obtaining, processing, synthesizing, storing and displaying a breast imaging data set or a subset thereof. In particular, the present disclosure relates to implementing one or more target object recognition/synthesis modules to identify respective objects in a tomosynthesis stack, and to combine results from the one or more target object recognition/synthesis modules to generate objects to display in one or more synthesized images.

### BACKGROUND

Mammography has long been used to screen for breast cancer and other abnormalities. Traditionally, mammograms have been formed on x-ray film. More recently, flat panel digital imagers have been introduced that acquire a mammogram in digital form, and thereby facilitate analysis and storage of the acquired image data, and to also provide other benefits. Further, substantial attention and technological development have been dedicated to obtaining three-dimensional images of the breast using methods such as breast tomosynthesis. In contrast to the 2D images generated by legacy mammography systems, breast tomosynthesis systems construct a 3D image volume from a series of 2D projection images, each projection image obtained at a different angular displacement of an x-ray source relative to the image detector as the x-ray source is scanned over the detector. The constructed 3D image volume is typically presented as a plurality of slices of image data, the slices being mathematically reconstructed on planes typically parallel to the imaging detector. The reconstructed tomosynthesis slices reduce or eliminate the problems caused by tissue overlap and structure noise present in single slice, two-dimensional mammography imaging, by permitting a user (e.g., a radiologist or other medical professional) to scroll through the image slices to view only the structures in that slice.

Imaging systems such as tomosynthesis systems have recently been developed for breast cancer screening and diagnosis. In particular, Hologic, Inc. (www.hologic.com) has developed a fused, multimode mammography/tomosynthesis system that acquires one or both types of mammogram and tomosynthesis images, either while the breast remains immobilized or in different compressions of the breast. Other companies have introduced systems that include tomosynthesis imaging; e.g., which do not include the ability to also acquire a mammogram in the same compression.

Examples of systems and methods that leverage existing medical expertise in order to facilitate, optionally, the transition to tomosynthesis technology are described in U.S. Pat. No. 7,760,924, which is hereby incorporated by reference in its entirety. In particular, U.S. Pat. No. 7,760,924 describes a method of generating a synthesized 2D image, which may optionally be displayed along with tomosynthesis projection or reconstructed images, in order to assist in screening and diagnosis.

The 2D synthesized image is designed to provide a concise representation of the 3D reconstruction slices, including any clinically important and meaningful information, such as abnormal lesions and normal breast structures, while representing in relevant part a traditional 2D image. There are many different types of lesions and breast structures, which may be defined as different types of image objects having different characteristics. For any given image object visible in the 3D volume data, it is important to maintain and enhance the image characteristics (e.g., micro-calcifications, architectural distortions, etc.) as much as possible onto the 2D synthesized image. To achieve the enhancement of the targeted image object, it is critical to accurately identify and represent the image object present in the 3D tomosynthesis data.

### SUMMARY

In one embodiment of the disclosed inventions, a method for processing breast tissue image data includes obtaining image data of a patient's breast tissue, processing the image data to generate a set of image slices, the image slices collectively depicting the patient's breast tissue; feeding image slices of the set through each of a plurality of object-recognizing modules, each of the object-recognizing modules being configured to recognize a respective type of object that may be present in the image slices; combining objects recognized by the respective object-recognizing modules to generate a synthesized image of the patient's breast tissue; and displaying the synthesized image.

These and other aspects and embodiments of the disclosed inventions are described in more detail below, in conjunction with the accompanying figures.

### BRIEF DESCRIPTION OF THE FIGURES

The drawings illustrate the design and utility of embodiments of the disclosed inventions, in which similar elements are referred to by common reference numerals. These drawings are not necessarily drawn to scale. In order to better appreciate how the above-recited and other advantages and objects are obtained, a more particular description of the embodiments will be rendered, which are illustrated in the accompanying drawings. These drawings depict only typical embodiments of the disclosed inventions and are not therefore to be considered limiting of its scope.

FIG. 1 is a block diagram illustrating the flow of data through an exemplary breast image acquisition and processing system in accordance with embodiments of the disclosed inventions;

FIG. 2 is a block diagram illustrating the flow of data through a 2D synthesizer that utilizes multiple target object recognition/enhancement modules to identify respective objects in an image stack in accordance with embodiments of the disclosed inventions;

FIG. 3 illustrates one embodiment of applying target object recognition/enhancement modules on an image stack to recognize respective objects and reduce the objects onto the 2D synthesized image;

FIG. 4 illustrates a flow of data when applying a single target object recognition/enhancement module on an image stack;

FIG. 5 illustrates a flow of data when applying multiple target object recognition/enhancement modules on an image stack;

FIGS. 6A and 6B illustrate a sequential combination technique of combining data from multiple target object synthesis modules;

FIGS. 7A and 7B illustrate a parallel combination technique of combining data from multiple target object synthesis modules; and

FIGS. 8A and 8B illustrate two example flow diagrams of generating 2D synthesized images using the sequential combination and parallel combination techniques respectively.

### DETAILED DESCRIPTION OF THE ILLUSTRATED EMBODIMENTS

All numeric values are herein assumed to be modified by the terms “about” or “approximately,” whether or not explicitly indicated, wherein the terms “about” and “approximately” generally refer to a range of numbers that one of skill in the art would consider equivalent to the recited value (i.e., having the same function or result). In some instances, the terms “about” and “approximately” may include numbers that are rounded to the nearest significant figure. The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. In describing the depicted embodiments of the disclosed inventions illustrated in the accompanying figures, specific terminology is employed for the sake of clarity and case of description. However, the disclosure of this patent specification is not intended to be limited to the specific terminology so selected, and it is to be understood that each specific element includes all technical equivalents that operate in a similar manner. It is to be further understood that the various elements and/or features of different illustrative embodiments may be combined with each other and/or substituted for each other wherever possible within the scope of this disclosure and the appended claims.

Various embodiments of the disclosed inventions are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the disclosed inventions, which is defined only by the appended claims and their equivalents. In addition, an illustrated embodiment of the disclosed inventions needs not have all the aspects or advantages shown. For example, an aspect or an advantage described in conjunction with a particular embodiment of the disclosed inventions is not necessarily limited to that embodiment and can be practiced in any other embodiments even if not so illustrated.

For the following defined terms and abbreviations, these definitions shall be applied throughout this patent specification and the accompanying claims, unless a different definition is given in the claims or elsewhere in this specification:

An “acquired image” refers to an image generated while visualizing a patient's tissue. Acquired images can be generated by radiation from a radiation source impacting on a radiation detector disposed on opposite sides of a patient's tissue, as in a conventional mammogram.

A “reconstructed image” refers to an image generated from data derived from a plurality of acquired images. A reconstructed image simulates an acquired image not included in the plurality of acquired images.

A “synthesized image” refers to an artificial image generated from data derived from a plurality of acquired and/or reconstructed images. A synthesized image includes elements (e.g., objects and regions) from the acquired and/or reconstructed images, but does not necessarily correspond to an image that can be acquired during visualization. Synthesized images are constructed analysis tools.

An “Mp” image is a conventional mammogram or contrast enhanced mammogram, which are two-dimensional (2D) projection images of a breast, and encompasses both a digital image as acquired by a flat panel detector or another imaging device, and the image after conventional processing to prepare it for display (e.g., to a health professional), storage (e.g., in the PACS system of a hospital), and/or other use.

A “Tp” image is an image that is similarly two-dimensional (2D), but is acquired at a respective tomosynthesis angle between the breast and the origin of the imaging x rays (typically the focal spot of an x-ray tube), and encompasses the image as acquired, as well as the image data after being processed for display, storage, and/or other use.

A “Tr” image is a type (or subset) of a reconstructed image that is reconstructed from tomosynthesis projection images Tp, for example, in the manner described in one or more of U.S. Pat. Nos. 7,577,282, 7,606,801, 7,760,924, and 8,571,289, the disclosures of which are fully incorporated by reference herein in their entirety, wherein a Tr image represents a slice of the breast as it would appear in a projection x ray image of that slice at any desired angle, not only at an angle used for acquiring Tp or Mp images.

An “Ms” image is a type (or subset) of a synthesized image, in particular, a synthesized 2D projection image that simulates mammography images, such as a craniocaudal (CC) or mediolateral oblique (MLO) images, and is constructed using tomosynthesis projection images Tp, tomosynthesis reconstructed images Tr, or a combination thereof. Ms images may be provided for display to a health professional or for storage in the PACS system of a hospital or another institution. Examples of methods that may be used to generate Ms images are described in the above-incorporated U.S. Pat. Nos. 7,760,924 and 8,571,289.

It should be appreciated that Tp, Tr, Ms and Mp image data encompasses information, in whatever form, that is sufficient to describe the respective image for display, further processing, or storage. The respective Mp, Ms. Tp and Tr images are typically provided in digital form prior to being displayed, with each image being defined by information that identifies the properties of each pixel in a two-dimensional array of pixels. The pixel values typically relate to respective measured, estimated, or computed responses to X-rays of corresponding volumes in the breast, i.e., voxels or columns of tissue. In a preferred embodiment, the geometry of the tomosynthesis images (Tr and Tp) and mammography images (Ms and Mp) are matched to a common coordinate system, as described in U.S. Pat. No. 7,702,142. Unless otherwise specified, such coordinate system matching is assumed to be implemented with respect to the embodiments described in the ensuing detailed description of this patent specification.

The terms “generating an image” and “transmitting an image” respectively refer to generating and transmitting information that is sufficient to describe the image for display. The generated and transmitted information is typically digital information.

In order to ensure that a synthesized 2D image displayed to an end-user (e.g., an Ms image) includes the most clinically relevant information, it is necessary to detect and identify three-dimensional (3D) objects, such as malignant breast mass, tumors, etc., within the breast tissue. Towards this end, in accordance with embodiments of the presently disclosed inventions, 3D objects may be identified using multiple target object recognition/synthesis modules, wherein each target recognition/synthesis module may be configured to identify and reconstruct a particular type of object. These multiple target synthesis modules may work together in combining information pertaining to respective objects during the reconstruction process of generating one or more synthesized 2D images, ensuring that each object is represented accurately, and preserving clinically significant information on the 2D synthesized images that are the displayed to the end-user.

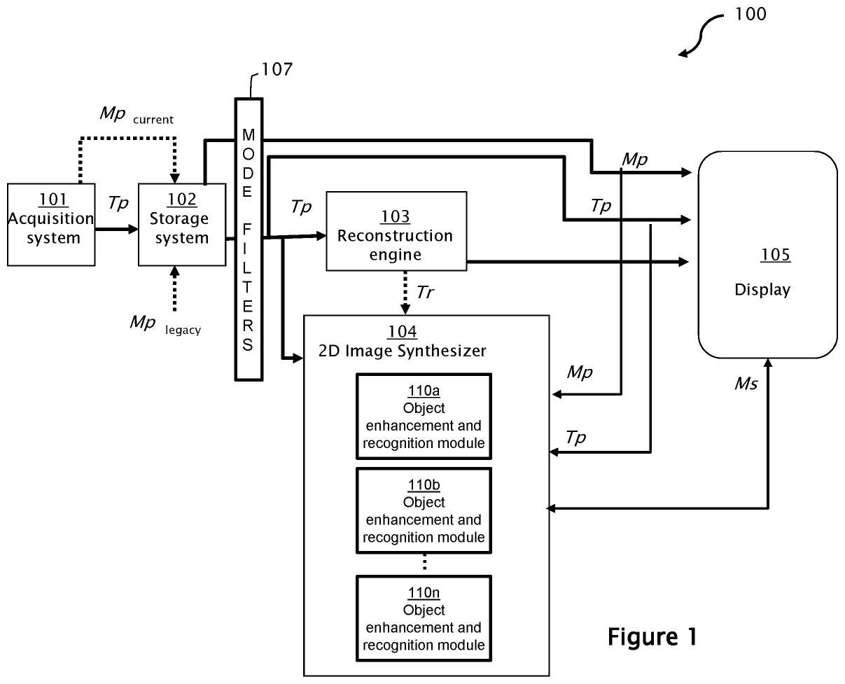

FIG. 1 illustrates the flow of data in an exemplary image generation and display system 100, which incorporates each of synthesized image generation, object identification, and display technology. It should be understood that, while FIG. 1 illustrates a particular embodiment of a flow diagram with certain processes taking place in a particular serial order or in parallel, the claims and various other embodiments described herein are not limited to the performance of the image processing steps in any particular order, unless so specified.

More particularly, the image generation and display system 100 includes an image acquisition system 101 that acquires tomosynthesis image data for generating Tp images of a patient's breasts, using the respective three-dimensional and/or tomosynthesis acquisition methods of any of the currently available systems. If the acquisition system is a combined tomosynthesis/mammography system, Mp images may also be generated. Some dedicated tomosynthesis systems or combined tomosynthesis/mammography systems may be adapted to accept and store legacy mammogram images, (indicated by a dashed line and legend “Mplegacy” in FIG. 1) in a storage device 102, which is preferably a DICOM-compliant Picture Archiving and Communication System (PACS) storage device. Following acquisition, the tomosynthesis projection images Tp may also be transmitted to the storage device 102 (as shown in FIG. 1). The storage device 102 may further store a library of known 3D objects that may be used to identify significant 3D image patterns to the end-user. In other embodiments, a separate dedicated storage device (not shown) may be used to store the library of known 3D objects with which to identify 3D image patterns or objects.

The Tp images are transmitted from either the acquisition system 101, or from the storage device 102, or both, to a computer system configured as a reconstruction engine 103 that reconstructs the Tp images into reconstructed image “slices” Tr, representing breast slices of selected thickness and at selected orientations, as disclosed in the above-incorporated patents and applications.

Mode filters 107 are disposed between image acquisition and image display. The filters 107 may additionally include customized filters for each type of image (i.e., Tp, Mp, and Tr images) arranged to identify and highlight certain aspects of the respective image types. In this manner, each imaging mode can be tuned or configured in an optimal way for a specific purpose. For example, filters programmed for recognizing objects across various 2D image slices may be applied in order to detect image patterns that may belong to a particular high-dimensional objects. The tuning or configuration may be automatic, based on the type of the image, or may be defined by manual input, for example through a user interface coupled to a display. In the illustrated embodiment of FIG. 1, the mode filters 107 are selected to highlight particular characteristics of the images that are best displayed in respective imaging modes, for example, geared towards identifying objects, highlighting masses or calcifications, identifying certain image patterns that may be constructed into a 3D object, or for creating 2D synthesized images (described below). Although FIG. 1 illustrates only one mode filter 107, it should be appreciated that any number of mode filters may be utilized in order to identify structures of interest in the breast tissue.

The imaging and display system 100 further includes a 2D image synthesizer 104 that operates substantially in parallel with the reconstruction engine 103 for generating 2D synthesized images using a combination of one or more Tp, Mp, and/or Tr images. The 2D image synthesizer 104 consumes a set of input images (e.g., Mp, Tr and/or Tp images), determines a set of most relevant features from each of the input images, and outputs one or more synthesized 2D images. The synthesized 2D image represents a consolidated synthesized image that condenses significant portions of various slices onto one image. This provides an end-user (e.g., medical personnel, radiologist, etc.) with the most clinically-relevant image data in an efficient manner, and reduces time spent on other images that may not have significant data.

One type of relevant image data to highlight in the synthesized 2D images would be relevant objects found across one or more Mp, Tr and/or Tp images. Rather than simply assessing image patterns of interest in each of the 2D image slices, it may be helpful to determine whether any of the 2D image patterns of interest belong to a larger high-dimensional structure, and if so, to combine the identified 2D image patterns into a higher-dimensional structure. This approach has several advantages, but in particular, by identifying high-dimensional structures across various slices/depths of the breast tissue, the end-user may be better informed as to the presence of a potentially significant structure that may not be easily visible in various 2D slices of the breast.

Further, instead of identifying similar image patterns in two 2D slices (that are perhaps adjacent to each other), and determining whether or not to highlight image data from one or both of the 2D slices, identifying both image patterns as belonging to the same high-dimensional structure may allow the system to make a more accurate assessment pertaining to the nature of the structure, and consequently provide significantly more valuable information to the end-user. Also, by identifying the high-dimensional structure, the structure can be more accurately depicted on the synthesized 2D image. Yet another advantage of identifying high-dimensional structures within the various captured 2D slices of the breast tissue relates to identifying a possible size/scope of the identified higher-dimensional structure. For example, once a structure has been identified, previously unremarkable image patterns that are somewhat proximate to the high-dimensional structure may now be identified as belonging to the same structure. This may provide the end-user with an indication that the high-dimensional structure is increasing in size/scope.

To this end, the 2D image synthesizer 104 employs a plurality of target object recognition/enhancement modules (also referred to as target object synthesis modules) that are configured to identify and reconstruct different types of objects. Each target image recognition/synthesis module may be applied (or “run”) on a stack (e.g., a tomosynthesis image stack) of 2D image slices of a patient's breast tissue, and work to identify particular types of objects that may be in the breast tissue, and ensure that such object(s) are represented in a clinically-significant manner in the resulting 2D synthesized image presented to the end-user. For example, a first target image synthesis module may be configured to identify calcifications in the breast tissue. Another target image synthesis module may be configured to identify and reconstruct spiculated lesions in the breast tissue. Yet another target image synthesis module may be configured to identify and reconstruct spherical masses in the breast tissue. In one or more embodiments, the multiple target image synthesis modules process the image slice data and populate respective objects in a high-dimensional grid (e.g., 3D grid) comprising respective high-dimensional structures (e.g., 3D objects) present in the breast tissue. This high-dimensional grid may then be utilized to accurately depict the various structures in the 2D synthesized image.

A high-dimensional object may refer to any object that comprises at least three or more dimensions, e.g., 3D or higher object, or a 3D or higher object and time dimension, etc. Examples of such objects or structures include, without limitation, calcifications, spiculated lesions, benign tumors, irregular masses, dense objects, etc. An image object may be defined as a certain type of image pattern that exists in the image data. The object may be a simple round object in a 3D space, and a corresponding flat round object in a 2D space. It can be an object with complex patterns and complex shapes, and it can be of any size or dimension. The concept of an object may extend past a locally bound geometrical object. Rather, the image object may refer to an abstract pattern or structure that can exist in any dimensional shape. It should be appreciated that the inventions disclosed herein are not limited to 3D objects and/or structures, and may include higher-dimensional structures. It should be appreciated that each of the target image synthesis modules is configured for identifying and reconstructing respective types of objects. These “objects” may refer to 2D shapes, 2D image patterns, 3D objects, or any other high-dimensional object, but in any event will all be referred to as “objects” or “3D objects” herein for simplicity, but this illustrative use should not be otherwise read as limiting the scope of the claims.

In the illustrated embodiment, the 2D synthesizer 104 comprises a plurality of target object recognition/enhancement modules (e.g., 110a, 110b . . . 110n), each configured for recognizing and enhancing a particular type of object. Each of the target object recognition/enhancement modules 110 may be run on a 2D image stack (e.g., Tr image stack), and is configured to identify the respective object (if any is/are present) therein. By identifying the assigned object in the 2D image stack, each target object recognition/enhancement module 110 works to ensure that the respective object is preserved and depicted accurately in the resulting 2D synthesized image presented to the end-user.

In some embodiments, a hierarchical model may be utilized in determining which objects to emphasize or de-emphasize in the 2D synthesized image based on a weight or priority assigned to the target object recognition/enhancement module. In other embodiments, all objects may be treated equally, and different objects may be fused together if there is an overlap in the z direction, as will be discussed in further detail below. These reconstruction techniques allow for creation of 2D synthesized images that comprise clinically-significant information, while eliminating or reducing unnecessary or visually confusing information.

The synthesized 2D images may be viewed at a display system 105. The reconstruction engine 103 and 2D image synthesizer 104 are preferably connected to a display system 105 via a fast transmission link. The display system 105 may be part of a standard acquisition workstation (e.g., of acquisition system 101), or of a standard (multi-display) review station (not shown) that is physically remote from the acquisition system 101. In some embodiments, a display connected via a communication network may be used, for example, a display of a personal computer or of a so-called tablet, smart phone or other hand-held device. In any event, the display 105 of the system is preferably able to display respective Ms, Mp, Tr, and/or Tp images concurrently, e.g., in separate side-by-side monitors of a review workstation, although the invention may still be implemented with a single display monitor, by toggling between images.

Thus, the imaging and display system 100, which is described as for purposes of illustration and not limitation, is capable of receiving and selectively displaying tomosynthesis projection images Tp, tomosynthesis reconstruction images Tr, synthesized mammogram images Ms, and/or mammogram (including contrast mammogram) images Mp, or any one or sub combination of these image types. The system 100 employs software to convert (i.e., reconstruct) tomosynthesis images Tp into images Tr, software for synthesizing mammogram images Ms, software for decomposing 3D objects, software for creating feature maps and object maps. An object of interest or feature in a source image may be considered a ‘most relevant’ feature for inclusion in a 2D synthesized image based upon the application of the object maps along with one or more algorithms and/or heuristics, wherein the algorithms assign numerical values, weights or thresholds, to pixels or regions of the respective source images based upon identified/detected objects and features of interest within the respective region or between features. The objects and features of interest may include, for example, spiculated lesions, calcifications, and the like.

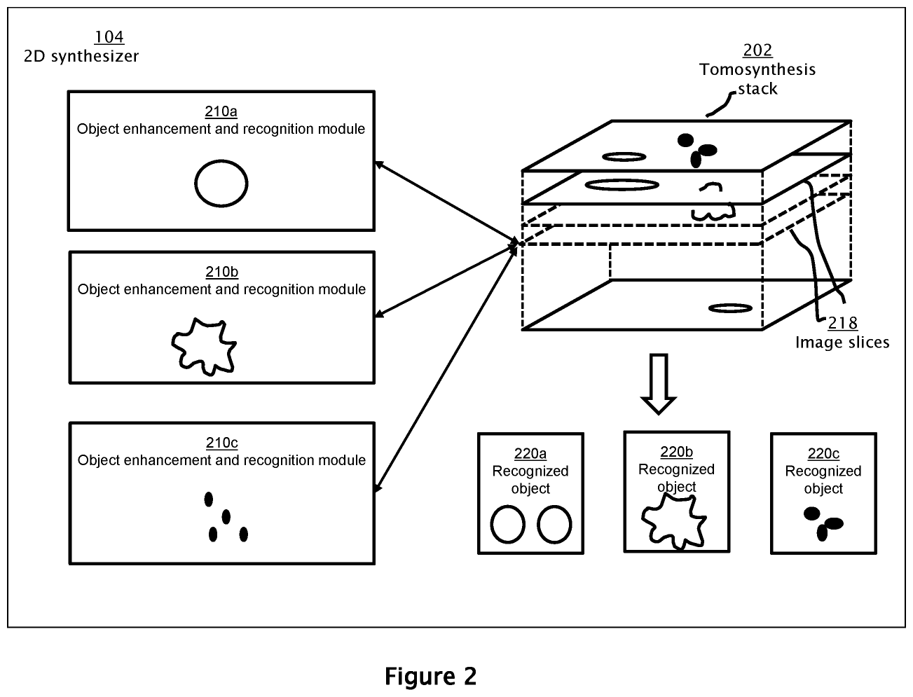

FIG. 2 illustrates the 2D image synthesizer 104 in further detail. As discussed above, various image slices 218 of a tomosynthesis data set (or “stack”) 202 (e.g., filtered and/or unfiltered Mp, Tr and/or Tp images of a patient's breast tissue) are input into the 2D image synthesizer 104, and then processed to determine portions of the images to highlight in a synthesized 2D image that will be displayed on the display 105. The image slices 218 may be consecutively-captured cross-sections of a patient's breast tissue. Or, the image slices 218 may be cross-sectional images of the patient's breast tissue captured at known intervals. The tomosynthesis image stack 202 comprising the image slices 218 may be forwarded to the 2D image synthesizer 104, which evaluates each of the source images in order to (1) identify various types of objects (Tr) for possible inclusion in one or more 2D synthesized images, and/or (2) identify respective pixel regions in the images that contain the identified objects.

As shown in the illustrated embodiment, the tomosynthesis stack 202 comprises a plurality of images 218 taken at various depths/cross-sections of the patient's breast tissue. Some of the images 218 in the tomosynthesis stack 202 comprise 2D image patterns. Thus, the tomosynthesis stack 202 comprises a large number of input images containing various image patterns within the images of the stack.

More particularly, as shown in FIG. 2, three target object recognition/enhancement modules 210a, 210b and 210c are configured to run on the tomosynthesis stack 202, wherein each of the target object recognition and enhancement modules 210 corresponds to a respective set of programs/rules and parameters that define a particular object, and how to identify that particular object amongst other objects that may exist in the breast tissue depicted by the tomosynthesis stack 202. For example, filtering/image recognition techniques and various algorithms/heuristics may be run on the tomosynthesis stack 202 in order to identify the object assigned to the particular target object recognition/enhancement module 210. It will be appreciated that there are many ways to recognize objects using a combination of image manipulation/filtration techniques.

For the purposes of illustration, it will be assumed that the each of the target object recognition/enhancement modules 210 identifies at least one respective object, but it should be appreciated that in many cases no objects will be identified. However, even healthy breast tissue may have one or more suspicious objects or structures, and the target object recognition/enhancement modules may inadvertently identify a breast background object. For example, all breast linear tissue and density tissue structures can be displayed as the breast background object. In other embodiments, “healthy” objects such as spherical shapes, oval shapes, etc., may simply be identified by one or more of the target object recognition/enhancement modules 210. The identified 3D objects may then be displayed on the 2D synthesized image 206; of course, out of all identified 2D objects, more clinically-significant objects may be prioritized/enhanced when displaying the respective objects on the 2D synthesized image, as will be discussed in further detail below.

In the illustrated embodiment, a first target object recognition/enhancement module 210a is configured to recognize circular and/or spherical shapes in the images 218 of the tomosynthesis stack 202 (e.g., Tr, Tp, Mp, etc.). A second target object synthesis module 210b is configured to recognize lobulated shapes. A third target object synthesis module 210c is configured to recognize calcification patterns. In particular, each of the target object synthesis modules 210a, 210b and 210c is run on the Tr image stack 202, wherein a set of features/objects are recognized by the respective target object synthesis modules.

For example, target object recognition/enhancement module 210a may recognize one or more circular shapes and store these as “recognized objects” 220a. It will be appreciated that multiple image slices 218 of the stack 202 may contain circular shapes, and that these shapes may be associated with the same spherical object, or may belong to different spherical objects. In the illustrated embodiment, at least two distinct circular objects are recognized by the target object recognition/enhancement module 210a.

Similarly, target object recognition/enhancement module 210b may recognize one or more lobulated shapes and store these as recognized objects 220b. In the illustrated embodiment, one lobulated object has been recognized in the tomosynthesis stack 202 by the target object recognition/enhancement module 210b. As can be seen, two different image slices 218 in the tomosynthesis stack 202 depict portions of the lobulated object, but the respective portions are recognized as belonging to a single lobulated object by the recognition/enhancement module 210b, and stored as a single recognized object 220b.

Finally, target object recognition/enhancement module 210c may recognize one or more calcification shapes and store these as recognized objects 220c. In the illustrated embodiment, a (single) calcification cluster has been recognized by the target object recognition/enhancement module 210c and stored as a recognized object 220c. The recognized objects 220a, 220b and 220c may be stored at storage facilities corresponding to the respective target object recognition/enhancement modules 210a, 210b and 210c, or alternatively at a separate (i.e., single) storage facility that may be accessed by each of the target object recognition/enhancement modules.

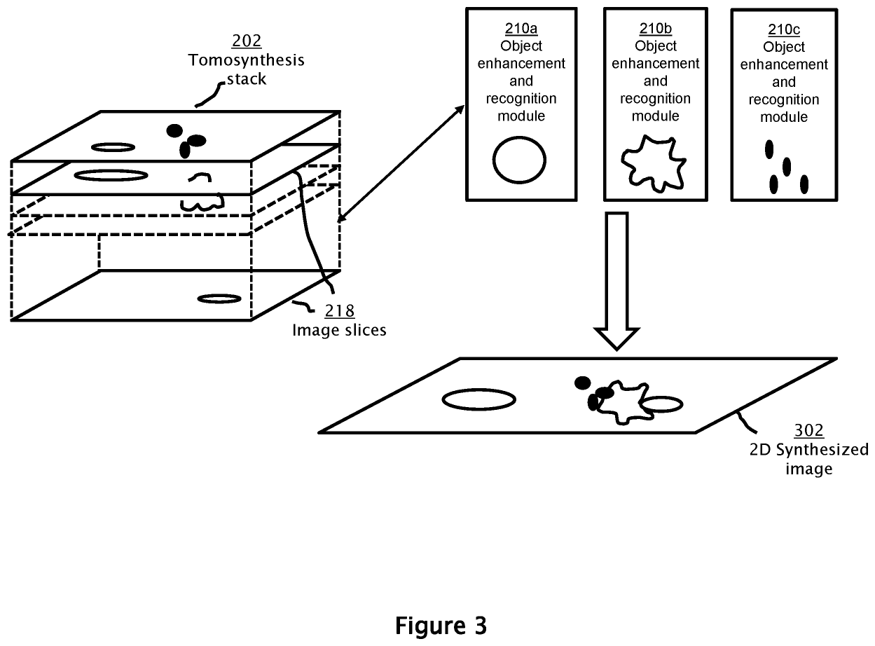

Referring now to FIG. 3, each of the target object recognition/enhancement modules 210 may be configured to identify and synthesize (e.g., to reduce to 2D) a respective 3D object to be displayed on the one or more 2D synthesized images. In other words, once the 3D objects are recognized by the respective target object recognition/enhancement module 210a, 210b or 210c, the target object recognition/enhancement module thereafter converts the recognized 3D object into a 2D format so that the recognized object may be displayed on the 2D synthesized image. In the illustrated embodiment, the target object recognition/enhancement modules 210a, 21b and 210c recognize respective objects, and convert the recognized objects into respective 2D formats. As part of the conversion process, certain of the recognized objects may be enhanced to a greater or lesser degree for the displayed image, as will be discussed in further detail below. Assuming all three target object recognition/enhancement modules 210a, 210b and 210c are considered equally important to the 2D image synthesizer 104, the respective 2D formats of all recognized objects (e.g., two spherical objects, one lobular object, and one calcification mass) depicted on the 2D synthesized image 302.

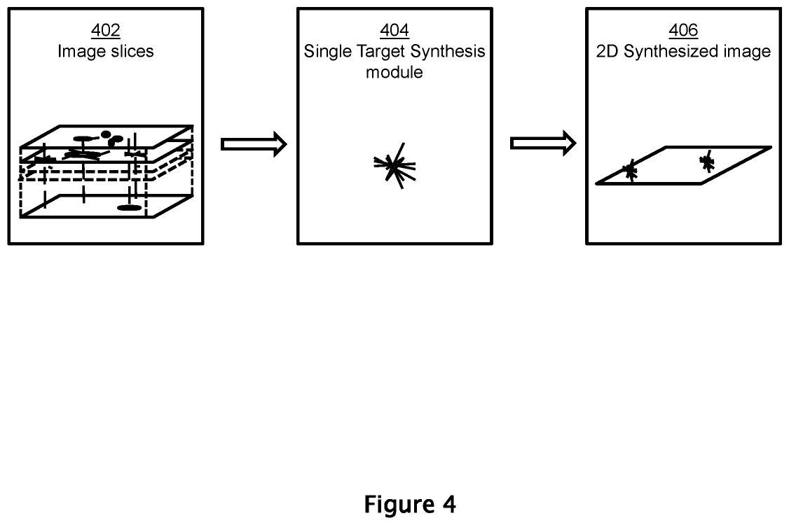

FIG. 4 illustrates how a single target object recognition/enhancement module 210 may be run on a tomosynthesis stack to generate a portion of the 2D synthesized image. In the illustrated embodiment, image slices 402 are fed through a single target object recognition/enhancement module 404, which is configured to recognize star shaped objects in the stack of images 402. As a result, the single target object synthesis module reduces information pertaining to the recognized star shape gained from various depths of the image slices onto a single 2D synthesized image 406.

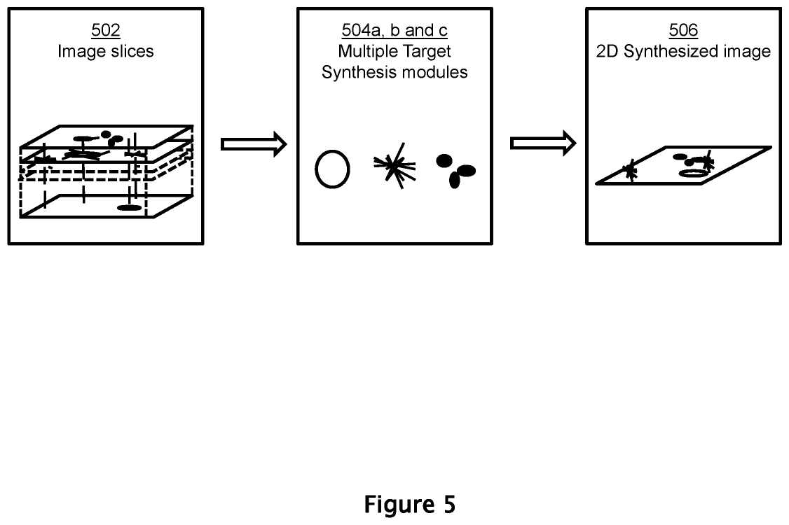

FIG. 5 illustrates an exemplary embodiment for having multiple target object recognition/enhancement modules work together to produce the 2D synthesized image. In the illustrated embodiment, image slices 502 (of a respective stack) are fed through a first target object recognition/enhancement module 504a configured to recognize and reconstruct circular and/or spherical shapes, a second target object recognition/enhancement module 504b configured to recognize and reconstruct star-like shapes, and a third target object recognition/enhancement module 504c configured to recognize and reconstruct calcification structures. It should be appreciated that any number of target object recognition/enhancement modules may be programmed for any number of object types.

Each of the target object recognition/enhancement modules 504a, 504b and 504c corresponds to respective algorithms that are configured with various predetermined rules and attributes that enable these programs to successfully recognize respective objects, and reduce the recognized objects to a 2D format. By applying all three target object recognition/synthesis modules 504a, 504b and 504c to the image slices 502, a 2D synthesized image 506 is generated. In particular, rather than simply displaying a single type of object, the 2D synthesized image 506 comprises all three object types that are recognized and synthesized by the three target object recognition/enhancement modules 504a, 504b and 504c, with each of the recognized objects being equally emphasized. While this may be desirable if all the object types are of equal significance, it may be helpful to enhance/emphasize different object types to varying degrees based on their weight/priority. This technique may be more effective in alerting the end-user to a potentially important object, while de-emphasizing objects of lesser importance.

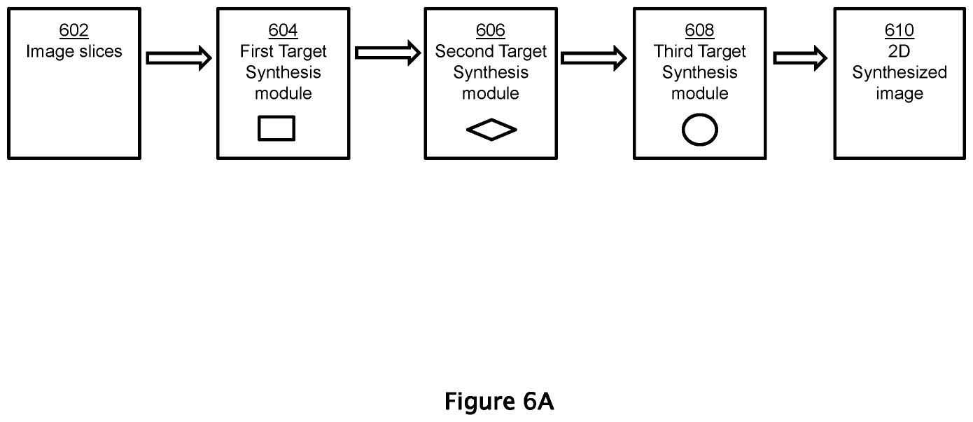

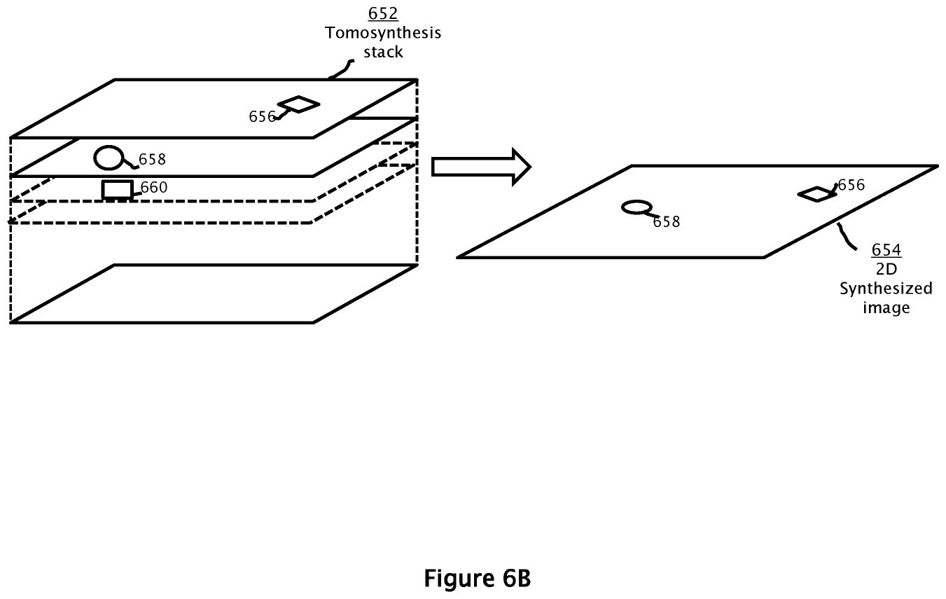

Referring now to FIG. 6A, a hierarchical sequential approach to combine data from the multiple target object recognition/enhancement modules is illustrated. In particular, a sequential combination technique may be applied if the various object types have a clearly defined hierarchy associated with them. For example, one type of object (e.g., spiculated lesions) may be deemed to be more clinically significant than another type of object (e.g., a spherical mass in breast tissue). This type of object (and the corresponding target object module) may be assigned a particular high weight/priority. In such a case, if two objects are competing for space on the 2D synthesized image, the object type associated with the higher priority may be emphasized/displayed on the 2D synthesized image, and the other object type may be de-emphasized, or not displayed at all. Similarly, in such an approach, each of the target object recognition/enhancement modules may be assigned respective weights based on respective significance.

In the illustrated embodiment, the image slices 602 are sequentially fed through three different target object recognition/enhancement modules (604, 606 and 608) to generate the 2D synthesized image 610, wherein each of the target object synthesis modules is configured to recognize and reconstruct a particular type of object. The first target object recognition/enhancement module 604 (associated with a square-shaped object) is run first on the reconstruction image slices 602, followed by the second target object recognition/enhancement module 606 (associated with a diamond-shaped object), and then followed by the third target object recognition/enhancement module 608 (associated with a circular-shaped object). It should be appreciated that since the target object recognition/enhancement modules are applied (or “run”) sequentially, the second target object recognition/enhancement module 606 may be considered a higher priority object as compared with the first target object recognition/enhancement module 604, and the third target object recognition/enhancement module 608 may be considered as having a higher priority as compared to the second target object recognition/enhancement module 606. Thus, the third object type may override (or be emphasized over) the second object type, and the second object type may override (or be emphasized over) the first object type.

FIG. 6B illustrates this hierarchical approach to combining various object types sequentially. In particular, the tomosynthesis image stack 652 includes objects 656, 658 and 660 that can be recognized in various image slices. As illustrated, objects 658 and 660 somewhat overlap in the z direction, which means that they are likely to compete for representation in the 2D synthesized image 654. When using the sequential approach of FIG. 6A to combine data from the multiple target object recognition/enhancement modules 604, 606 and 608, the programmed hierarchy is preserved. Thus, since target object recognition/enhancement module 608 configured to recognize and reconstruct circular-shaped objects has higher priority as compared to target object recognition/enhancement module 604 configured to recognize and reconstruct square-shaped objects, in a case of overlap between the two objects (as is the case in FIG. 6B), circular-shaped object 658 overrides square-shaped object 660 in the 2D synthesized image 654. Of course, it should be appreciated that since diamond-shaped object 656 does not overlap in the z direction with the other two objects, diamond shaped object 656 is also displayed in the 2D synthesized image 654. In other embodiments, instead of completing overriding the lower-priority object, the object with high-priority may be emphasized relative to the lower-priority object (rather than be omitted from display).

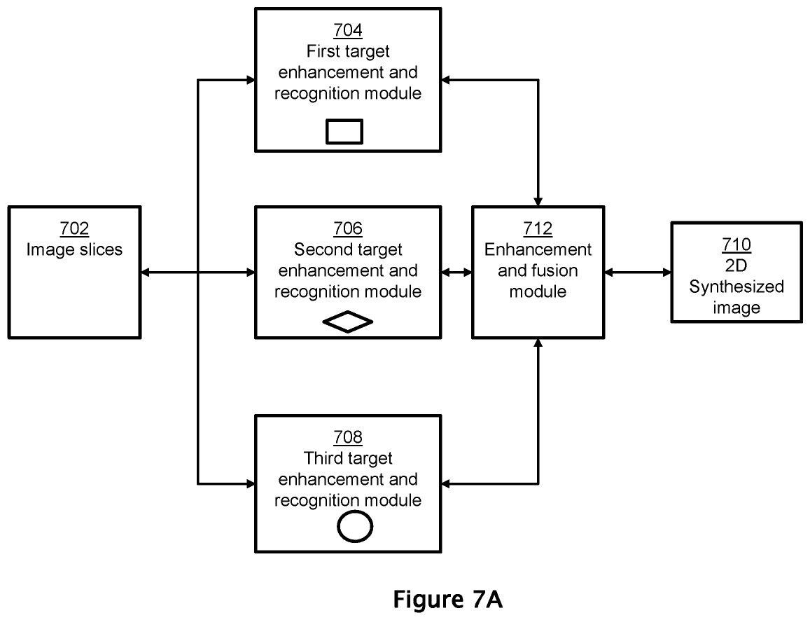

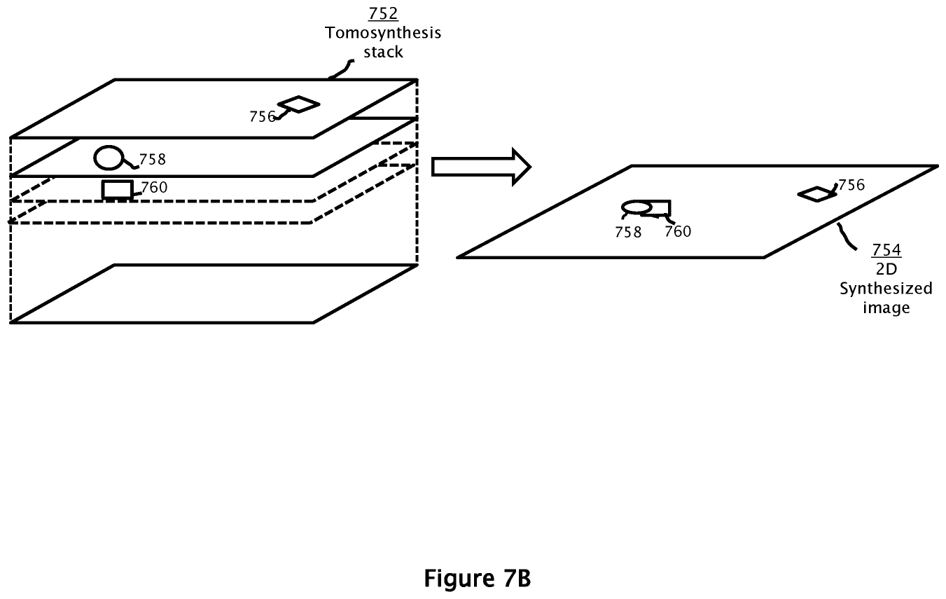

Another approach to running multiple target object synthesis modules on a set of image slices is illustrated in FIG. 7A. As can be seen, rather than running the multiple target object recognition/enhancement modules sequentially with the last-run target object synthesis module having the highest priority, all the target object recognition/enhancement modules may be applied in parallel. In particular, one or more enhancement or fusion modules 712 may be utilized to ensure that the various objects are combined appropriately on the 2D synthesized image. This approach may not follow a hierarchical approach, and all of the objects may be given equal weight.

The image slices 702 are fed through three different target object recognition/enhancement modules, 704, 706 and 708, in parallel. The first target object recognition/enhancement module 604 (associated with square-shaped object), the second target object recognition/enhancement module 606 (associated with diamond-shaped object), and the third target object recognition/enhancement module 608 (associated with circular-shaped object) are all run in parallel on the image slices 702. In some embodiments, an enhancement and fusion module 712 may be utilized to ensure that the different objects are fused together appropriately in case of overlap between multiple objects. The target object recognition/enhancement modules 704, 706 and 708, run in parallel may generate the 2D synthesized image 710.

This approach to combining various object types in parallel is illustrated in FIG. 7B. In particular, the tomosynthesis stack 752 depict the same objects as FIG. 6B (e.g., objects 756, 758 and 760) at various image slices. As illustrated, objects 758 and 760 somewhat overlap in the z direction, which means that they are likely to compete for representation and/or overlap in the 2D synthesized image 754. Here, because the multiple target object recognition/enhancement modules are run in parallel, rather than one object type overriding another object type, as was the case in FIG. 6B, both the square-object 760 and the circular object 758 are fused together in the 2D synthesized image 754. Thus, this approach does not assume an innate priority/hierarchy between objects and all objects may be fused together appropriately in the 2D synthesized image 754.

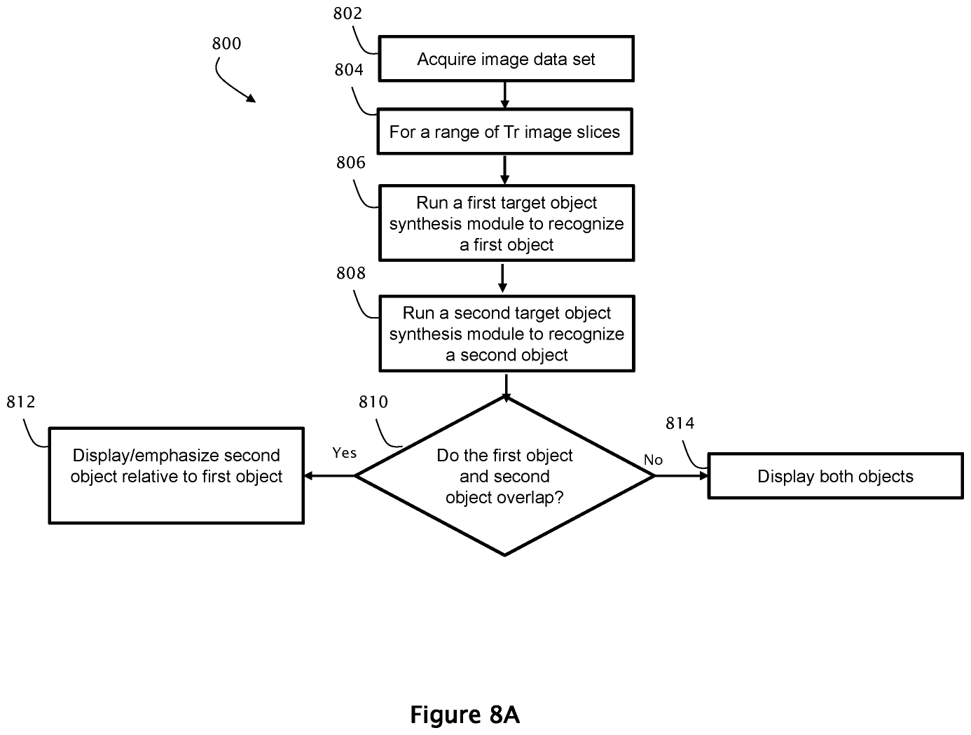

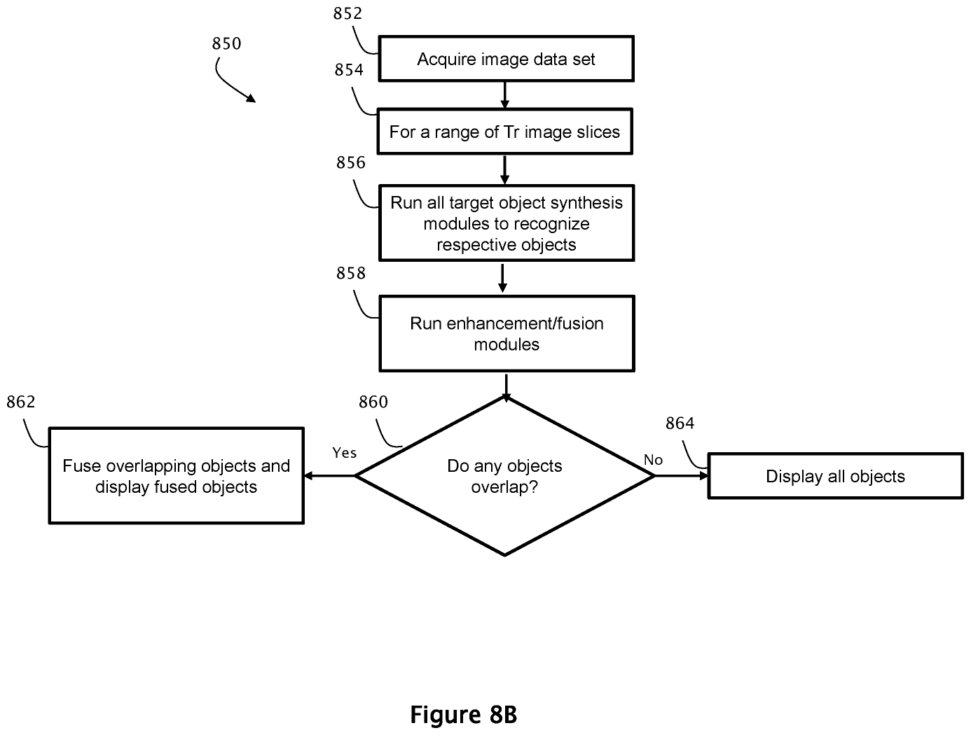

FIG. 8A depicts a flow diagram 800 that illustrates exemplary steps that may be performed in an image merge process carried out in accordance with the sequential combination approach outlined above in conjunction with FIGS. 6A and 6B. At step 802, an image data set is acquired. The image data set may be acquired by a tomosynthesis acquisition system, a combination tomosynthesis/mammography system, or by retrieving pre-existing image data from a storage device, whether locally or remotely located relative to an image display device. At steps 804 and 806, for a range of 2D images (e.g., Tr stack), a first target object recognition/enhancement module is run in order to recognize a first object associated with the first target object recognition/enhancement module. Any recognized objects may be stored in a storage module associated with the first target object recognition/enhancement module. At step 808, a second target object recognition/enhancement module is run in order to recognize a second object associated with the second target object recognition/enhancement module. At step 810, it may be determined whether the first recognize object and the second recognized object overlap each other in the z direction. If it is determined that the two objects overlap, only the second object may be displayed (or otherwise emphasized over the first object) on the 2D synthesized image at step 812. If, on the other hand, it is determined that the two objects do not overlap, both objects are displayed on the 2D synthesized image at step 814.

FIG. 8B depicts a flow diagram 850 that illustrates exemplary steps that may be performed in an image synthesis process carried out in accordance with the parallel combination approach outlined above in conjunction with FIGS. 7A and 7B. At step 852, an image data set is acquired. The image data set may be acquired by a tomosynthesis acquisition system, a combination tomosynthesis/mammography system, or by retrieving pre-existing image data from a storage device, whether locally or remotely located relative to an image display device. At steps 854 and 856, for a range of 2D images (e.g., Tr stack), all the programmed target object recognition/enhancement modules are run to recognize respective objects in the Tr image stack. At step 858, one or more enhancement modules may also be run to determine whether a fusion process needs to occur. At step 860, it may be determined whether any recognized objects overlap in the z direction. If it is determined that any two (or more) objects overlap, the overlapping objects may be fused together, at step 862. If, on the other hand, it is determined that no objects overlap, all the objects are displayed as is on the 2D synthesized image at step 814.

Having described exemplary embodiments, it can be appreciated that the examples described above and depicted in the accompanying figures are only illustrative, and that other embodiments and examples also are encompassed within the scope of the appended claims. For example, while the flow diagrams provided in the accompanying figures are illustrative of exemplary steps; the overall image merge process may be achieved in a variety of manners using other data merge methods known in the art. The system block diagrams are similarly representative only, illustrating functional delineations that are not to be viewed as limiting requirements of the disclosed inventions. It will also be apparent to those skilled in the art that various changes and modifications may be made to the depicted and/or described embodiments (e.g., the dimensions of various parts), without departing from the scope of the disclosed inventions, which is to be defined only by the following claims and their equivalents. The specification and drawings are, accordingly, to be regarded in an illustrative rather than restrictive sense.

1-20. (canceled)

21. A method for processing breast tissue image data, comprising:
processing image data of a patient's breast tissue to generate a set of image slices that collectively depict the patient's breast tissue, wherein at least one image slice in the set of image slices comprises an object of a particular object type;
performing a mode filter on the image slices to enhance the set of image slices;
performing a plurality of object-recognition processes on the image slices, wherein each of the plurality of object-recognition processes is configured to recognize a respective type of object that may be present in the image slices;
based at least in part on performing the plurality of object-recognition processes, recognizing the object of the particular object type in the at least one image slice in the set of image slices; and
generating, using the at least one image slice in the set of image slices, a synthesized image of the patient's breast tissue.

22. The method of claim 21, wherein the mode filter is based on an image type of the set of image slices

23. The method of claim 22, wherein the mode filter being based on an image type of the set of image slices comprises the mode filter being configured to highlight aspects of each image slice of the set of image slices based on the image type.

24. The method of claim 22, wherein the mode filter is automatically configured based on the image type.

25. The method of claim 24, wherein the mode filter comprises one or more filters.

26. The method of claim 24, wherein the mode filter is configured for at least one of identifying objects, highlighting masses or calcifications, identifying image patterns, and processing for generating a synthesized image.

27. The method of claim 22, wherein the mode filter is manually configured.

28. The method of claim 21, wherein performing the mode filter on the image slices occurs before performing the plurality of object-recognition processes on the images slices.

29. The method of claim 28, wherein the system is configured to allow for adding further object-recognition processes to the plurality of the object-recognition processes in order to recognize and display further types of objects.

30. The method of claim 28, wherein the image slices are fed to the respective object-recognizing modules in a sequence.

31. The method of claim 28, wherein the object-recognizing modules are applied in parallel on the image slices.

32. The method of claim 21, further comprising displaying target object types associated with the plurality of object-recognition processes in a graphical user interface.

33. The method of claim 32, wherein the graphical user interface provides options for an end user to select one or more target object types to be recognized and included in the synthesized image.

34. The method of claim 33, wherein the graphical user interface provides options for allowing an end user to input a weight factor for each of one or more target object types, and wherein user input weight factors are taken into account for generating and displaying user selected target object types in the synthesized image.

35. The method of claim 21, further comprising assigning a respective weight to each of the object-recognizing modules, wherein the assigned weight corresponds to a significance of the type of object recognized by the particular object-recognizing module, wherein the respective weights assigned to the object-recognizing modules determine an order of the object-recognizing modules through which the image slices are fed.

36. The method of claim 35, wherein the image slices are fed through a first object-recognizing module having a first weight before being fed through a second object-recognizing module having a second weight higher than the first weight.

37. The method of claim 36, further comprising determining whether an object of a first object type recognized by the first object-recognizing module and an object of a second type recognized by the second object-recognizing module are likely to overlap on the displayed synthesized image.

38. The method of claim 37, further comprising, if it is determined that the recognized object of the first object type and the recognized object of the second object type are likely to overlap, emphasizing the recognized object of the second object type relative to the recognized object of the first object type in the displayed synthesized image.

39. The method of claim 21, further comprising storing the synthesized image.

40. The method of claim 21, further comprising displaying the synthesized image.
